(19) United States
(12) Patent Application Publication
(10) Pub. No.: US 2004/0220557 A1
(43) Pub. Date: Nov. 4, 2004

(54) Closed system warming catheter and method of use

(76) Inventors: Jay J. Eum, Irvine, CA (US); Thach Duong, Garden Grove, CA (US)

Correspondence Address: LAWRENCE N. GINSBERG, ENDOCARE, INC., 201 TECHNOLOGY DRIVE, IRVINE, CA, 92618, US

(21) Appl. No.: 10/426,807

(22) Filed: Apr. 30, 2003

(51) Int. Cl. A61B 18/18

(52) U.S. Cl. 606/21

(57) ABSTRACT

An apparatus and method for warming the urethra of a patient during ablative surgery. In one embodiment, at least one ablative surgical device is inserted into a prostate region of the patient. A Joule-Thomson warming assembly is inserted through the patient's urethra and into the bladder. The Joule-Thomson warming assembly is operated to warm an outer surface thereof during operation of the ablative surgical devices. The urethra is warmed by the outer surface of the Joule-Thomson warming assembly to preserve living tissue thereof.

In another embodiment the portion inserted through the patient's urethra is an electrical coil heated warming catheter subassembly. In another embodiment the inserted portion is a microwave heated tube warming catheter subassembly. Another embodiment comprises an RF heated warming catheter subassembly.

An opening is preferably provided to provide access for an endoscope or for fluid drainage.

### BACKGROUND OF THE INVENTION

1. Field of the Invention

The present invention relates to urological warming and cooling devices and more particularly to a warming catheter and method of warming the urethra of a patient during ablative surgery.

2. Description of the Related Art

Cryosurgical probes are used to treat a variety of diseases. The cryosurgical probes quickly freeze diseased body tissue, causing the tissue to die after which it will be absorbed by the body, expelled by the body or sloughed off. Cryothernal treatment is currently used to treat prostate cancer and benign prostate disease, breast tumors and breast cancer, liver tumors and liver cancer, glaucoma and other eye diseases. Cryosurgery is also proposed for the treatment of a number of other diseases.

The use of cryosurgical probes for cryoablation of the prostate is described in, for example, Onik, Ultrasound-Guided Cryosurgery, Scientific American at 62 (January 1996). Cryosurgical probe systems are manufactured by present assignee, Endocare, Inc. of Irvine, Calif. In cryosurgical ablation procedures generally several cryosurgical probes are inserted through the skin in the perineal area (between the scrotum and the anus), which provides the easiest access to the prostate. The probes are pushed into the prostate gland through previously placed cannulas. Placement of the probes within the prostate gland is typically visualized with an ultrasound imaging probe placed in the rectum. The probes are quickly cooled to temperatures typically below −120° C. The prostate tissue is killed by the freezing, and any tumor or cancer within the prostate is also killed. The body absorbs some of the dead tissue over a period of several weeks. However, other necrosed tissue may slough off and pass through the urethra, often causing undesirable blockage. Thus, it is often desirable to avoid cryoinjury to the urethra during cryoablation of the prostate. This may be done by placing a warming catheter in the urethra and continuously flushing the catheter with warm fluid to keep the urethra from freezing.

Devices for warming the urethra have been available for quite some time. In 1911, U.S. Pat. No. 1,011,606 issued for an “Appliance For Subjecting Portions Of The Human System To Heat Or Cold.” This device was a coaxial dual lumen catheter intended for the application of therapeutic cooling or heating to the urethra and bladder. Devices for warming other body parts have also been proposed, such as U.S. Pat. No. 4,244,377, issued Jan. 13, 1981 to Grams entitled “Ear Probe For Use In Closed-Loop Caloric Irrigation”, which shows a coaxial dual lumen cannula intended for the application of therapeutic heating inside the ear.

U.S. Pat. No. 5,437,673, issued on Aug. 1, 1995 to Baust, et al., entitled “Closed Circulation Tissue Warming Apparatus and Method of Using the Same in Prostate Surgery” illustrate use of a urethral warming catheter which is used to protect the urethra from cryothermal damage during cryosurgical treatment of the prostate for benign prostate hyperplasia. The Baust patent discloses a coaxial three lumen catheter in which warm saline passes through an outside lumen and is returned through a coaxial second lumen. A third lumen is a urinary drainage lumen centrally disposed within the other two lumens. The catheter is used to heat the urethra while the prostate is being frozen with cryosurgical probes.

U.S. Pat. No. 5,257,977, issued on Nov. 2, 1993 to Eshel, entitled “Technique for Localized Thermal Treatment of Mammals,” shows a catheter that delivers heated saline flow to provide therapeutic hyperthermia treatment of the prostate. Like the Baust patent, Eshel shows a three lumen catheter with a centrally located urinary drainage lumen.

Still other devices have been described for importing fluid into the body and allowing a means for removing fluid from the body. One such device is described in U.S. Pat. No. 3,087,493, issued Apr. 27, 1960 to Schossow, entitled “Endotracheal Tube”. Schossow describes a device employed to intubate the human trachea. The device is connected with ducts and/or tubes outside the patient for the purpose of, for example, drawing off from the patient's respiratory tract undesirable liquids and/or introducing beneficial liquids into the trachea. The device comprises an outer tube, which fits inside the patient's trachea, and a two layered inner tube. The lumen of the inner tube is open to be connected with devices or ducts through which suction may be applied or fluids injected into the trachea. The distal portion of the inner tube is vented with ports or openings that create a “sprinkler” effect inside the tube.

During cryoablation, the prostate tissue is killed by freezing temperatures in the cryogenic temperature range, typically −120° C. and below. The hot fluid used for the warming catheter is supplied at about 30° C. to 50° C. Warm fluid is pumped through the urethral warming catheter, such as the catheter described in Baust et al. Using this catheter, as the warm fluid travels the length of the urethral catheter disposed within the cryosurgically-cooled urethra, it is cooled by the surrounding freezing tissue. By the time the hot water has traveled from the bladder neck sphincter to the external sphincter, it has been significantly cooled by the surrounding frozen prostate. As a result, the urethral tissue near the bladder neck sphincter (near the hot water outlet) is heated more than the urethral tissue near the external sphincter, creating a strong thermal gradient in the prostatic urethra and an uneven heating effect. By the time the hot water reaches the external sphincter, it may have lost so much heat to the upper region of the urethra that it is not warm enough to protect the external sphincter from freezing. In order for the tissue at the bladder neck sphincter to be adequately warmed, hotter water must be pumped in, risking urethral damage due to scalded tissue, or more water must be pumped at higher rates and pressures, increasing the material requirements of the hot water supply system and the warming catheter.

U.S. Pat. No. 6,017,361, issued to Mikus et al, entitled Urethral Warming Catheter, discloses an improved method and means for maintaining the temperature of urethral tissues during cryoablation of the prostate gland and thereby eliminates or reduces the sloughing of dead cells into the urethra. Diffuser holes or ports, much like a “sprinkler,” are drilled into the inner tube of the warming catheter. The holes create an advantage over the prior art of achieving improved uniformity of fluid flow and temperature, utilizing a lower initial temperature and resulting in a more even application of thermal treatment to the urethral tissues. The apparatus may find additional utility in other areas of surgery where thermal treatment or maintenance of tissues is required with or without the capability of drainage.

U.S. Pat. No. 6,067,475, issued to Kenneth L. Graves et al, entitled Microwave Energy Delivery System Including High Performance Dual Directional Coupler for Precisely Measuring Forward and Reverse Microwave Power During Thermal Therapy, discloses a microwave energy delivery system for microwave thermal therapy that includes an antenna and a transmission line connected to the antenna. A microwave generating source includes a generator connected to the transmission line and a dual directional coupler for detecting forward power delivered to the antenna and reverse power reflected from the antenna with low uncertainty.

### SUMMARY OF THE INVENTION

In one broad aspect the present invention is a method for warming the urethra of a patient during ablative surgery. In this method, at least one ablative surgical device is inserted into a prostate region of the patient. A Joule-Thomson warming assembly is inserted through the patient's urethra and at least to the bladder neck. The Joule-Thomson warming assembly is operated to warm an outer surface thereof during operation of the ablative surgical devices. The urethra is warmed by the outer surface of the Joule-Thomson warming assembly to preserve living tissue thereof.

The present invention is particularly advantageous for use during cryosurgical ablation of the prostate utilizing cryosurgical probes. In one implementation, the Joule-Thomson warming assembly comprises a Joule-Thomson warming subassembly with a central opening. The central opening can accommodate, for example, a drainage tube or an endoscope.

In another broad aspect, instead of utilizing Joule-Thomson warming, an electrically generated warming assembly is inserted through the patient's urethra and at least to the bladder neck. In one implementation an electrical coil heated warming catheter subassembly is utilized. In another implementation the electrically generated warming assembly comprises a microwave heated warming catheter subassembly. In another implementation the electrically generated warming assembly comprises an RF heated warming catheter subassembly.

### BRIEF DESCRIPTION OF THE DRAWINGS

FIG. 1 is a cross-sectional view of the lower abdominal portion of the human body with a warming assembly of the present invention in place.

FIG. 2 is a perspective view of an embodiment of the warming assembly in which a Joule-Thomson warming assembly is utilized.

FIG. 3 is an enlarged cross-sectional view of the distal portion of the warming assembly of FIG. 2.

FIG. 4 is a cross-sectional view of another embodiment of the warming assembly in which an electrically generated warming assembly is utilized.

FIG. 5 is an enlarged cross-sectional view of the distal portion of the warming assembly of FIG. 4.

FIG. 6 is a cross-sectional view of another embodiment of the warming assembly in which a microwave warming assembly is utilized.

FIG. 7 is a cross-sectional view of another embodiment of the warming assembly in which an RF warming assembly is utilized.

FIG. 8a is an enlarged perspective view, partially in section, of a portion of the warming assembly of FIG. 7.

FIG. 8b is a view taken along line 8b-8b of FIG. 8a.

### DETAILED DESCRIPTION OF THE PREFERRED EMBODIMENTS

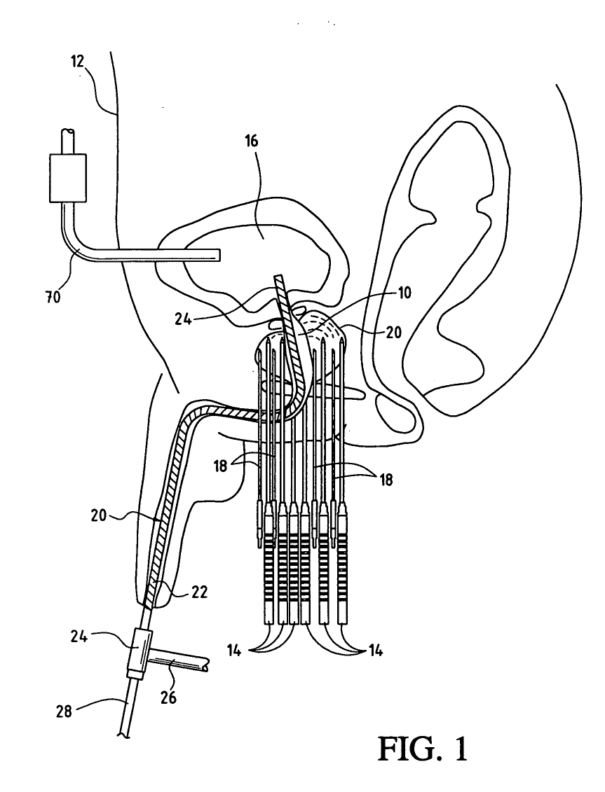

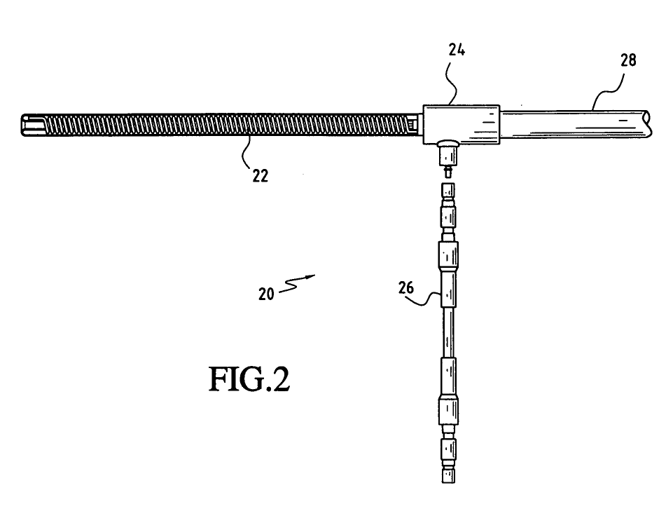

Referring now to the drawings and the characters of reference marked thereon, FIGS. 1-3 illustrate a first preferred method of warming a urethra 10 of a patient 12 during ablative surgery in accordance with the principles of the present invention. Ablative devices 14 are inserted into the prostate region 16 of the patient 12. A warming assembly, designated generally as 18, is inserted through the patient's urethra 10 at least to the bladder neck and generally into the bladder 16. Warming fluid is delivered through the warming assembly 20 during operation of the ablative surgical devices 18. The warming fluid is delivered into the bladder 16. The urethra is warmed by the warming fluid to preserve living tissue thereof.

The ablative devices are preferably cryosurgical probes such as manufactured and marketed by Endocare, Inc., of Irvine, Calif. The figure shows use of six cryosurgical probes 14 as well as four temperature probes 18. Alternatively, other ablative devices may be used, for example, radio frequency electrodes, laser fibers, microwave catheters, or high-intensity focused ultrasound. In such instances the heat exchange fluid is cool so as to prevent the urethra from the heating by the ablative elements.

In this first embodiment illustrated, the warming assembly 20 is a Joule-Thomson warming assembly, including an insertable Joule-Thomson warming subassembly 22, a connector element 24, an inlet subassembly 26, and an outlet subassembly 28. The outlet subassembly 28 is preferably formed of a flexible material such as one of various suitable plastics, for example, polyethelene. The connector element 24 is a suitable rigid material such as polycarbonate. The inlet subassembly 26 is connected to a high pressure gas source (not shown), preferably helium. Other suitable gases may be utilized that heat while undergoing Joule-Thomson expansion.

Although not shown, the inlet subassembly 26 receives heat exchange fluid from a pump and warmer, which are, in turn, connected to a reservoir. Generally, the warming gas should be supplied at pressures that cannot result in Joule-Thomson warming to temperatures sufficient to thermally damage the urethra.

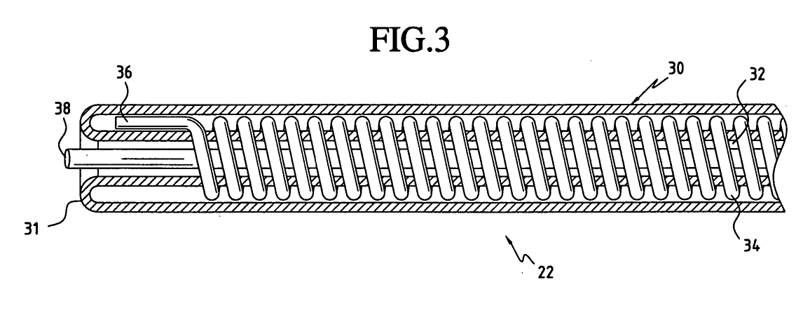

Referring now to FIG. 3, an enlarged view of an embodiment of the Joule-Thomson warming subassembly 22 is illustrated. It includes a tube assembly 30 having a closed distal end portion 31. The tube assembly 30 has an outer surface thereon for warming. The tube assembly 30 further includes an elongated opening 32 along a central axis of the tube assembly portion 30. A finned tube coiled heat exchanger 34 is disposed within the tube assembly 30. The heat exchanger 34 has a Joule-Thomson nozzle 36 on a distal end thereof and a high pressure gas inlet at a proximal end thereof. The finned tube coiled heat exchanger 34 has a plurality of windings with interstitial gaps between the windings to provide an outlet path for hot gas expelled from the Joule-Thomson nozzle 36. During operation, the windings provide heat transfer from the outlet path to inlet gases for enhanced efficiency. The central opening 32 is in fluid isolation from both the gases flowing in the finned tube coiled heat exchanger 34 and the outlet gases. The central opening 32 may provide, for example, access for an endoscope 38 and/or drainage for bladder fluid.

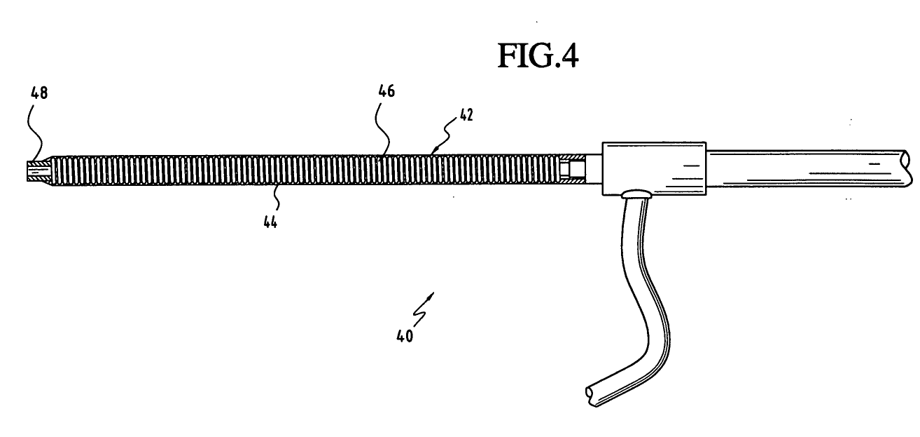

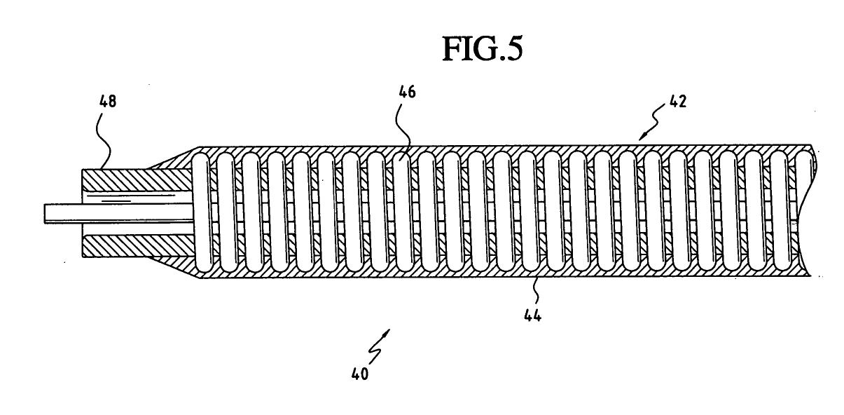

Referring now to FIGS. 4-5, another embodiment of the warming assembly is illustrated, designated generally as 40. In this embodiment the portion inserted through the patient's urethra is an electrical coil heated warming catheter subassembly 42. The electrical coil heated warming catheter subassembly 42 includes an electrical coil heated tube assembly 44 having a closed distal end portion. An electrical coil assembly 46 is disposed within the electrically heated tube assembly 44. The heating system used may be used such those marketed by, for example, Watlow Electric Manufacturing Company, 12001 Lackland Road, St. Louis, Mo. There are a number of potential candidate technologies that may be used to provide electrical heating such as multicell heaters, cable heaters, polymer heaters, cartridge heaters, radiant heaters, strip heaters, ceramic fiber heaters, thick film heaters, tubular heaters, and flexible heaters. U.S. Pat. No. 6,414,281 entitled “Hot-Toe Multicell Electric Heater”, discloses and claims a multicell heater having multiple heaters wrapped in a single tubing.

As in the previous embodiment the electrical coil heated warming catheter subassembly 42 preferably includes an elongated opening 48 to provide, for example, access for an endoscope and/or drainage for bladder fluid.

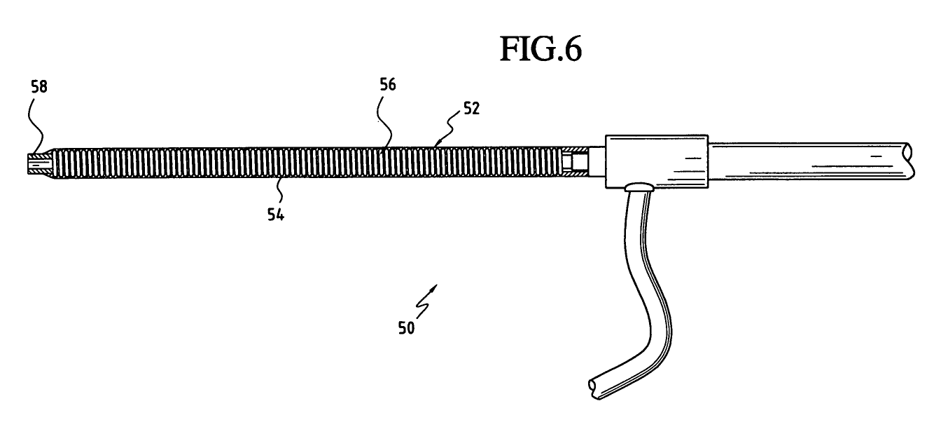

Referring now to FIG. 6, another embodiment of the warming assembly is illustrated, designated generally as 50. In this embodiment the portion inserted through the patient's urethra is a microwave heated tube warming catheter subassembly 52. The microwave heated warming catheter subassembly 52 includes a microwave heated tube assembly 54 having a closed distal end portion. A microwave generating assembly 56 is disposed within the microwave heated tube assembly 54. Microwave heating in the vicinity of the prostate is known. For example, U.S. Pat. No. 5,843,144, entitled “Method for Treating Benign Prostatic Hyperplasia With Thermal Therapy,” discloses a method for treating BPH with transurethral thermal ablation therapy.

U.S. Pat. No. 4,967,765 entitled “Urethral Inserted Applicator For Prostate Hyperthermia”, discloses another system for treating BPH. U.S. Pat. No. 5,249,585, entitled “Urethral Inserted Applicator for Prostate Hyperthermia,” discloses another such system. U.S. Pat. No. 5,509,929, entitled “Urethral Probe and Apparatus For the Therapeutic Treatment of the Prostate By Themotherapy,” discloses use of a microwave antenna directed onto the prostatic tissues located at least at the level of the bladder neck in the working position. U.S. Pat. No. 5,480,417, entitled “Method and Apparatus For the Surgical Treatment of Tissues by Thermal Effect, and in Particular the Prostate, Using a Urethral Microwave-Emitting Probe Means,” discloses another system for providing thermotherapy of the prostate.

As in the previous embodiments the microwave heated tube warming catheter subassembly 52 preferably includes an elongated opening 58 to provide, for example, access for an endoscope and/or drainage for bladder fluid.

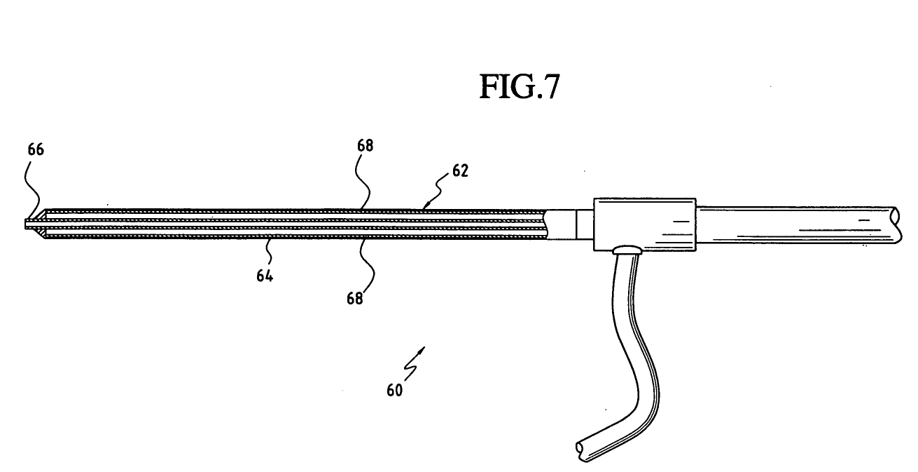

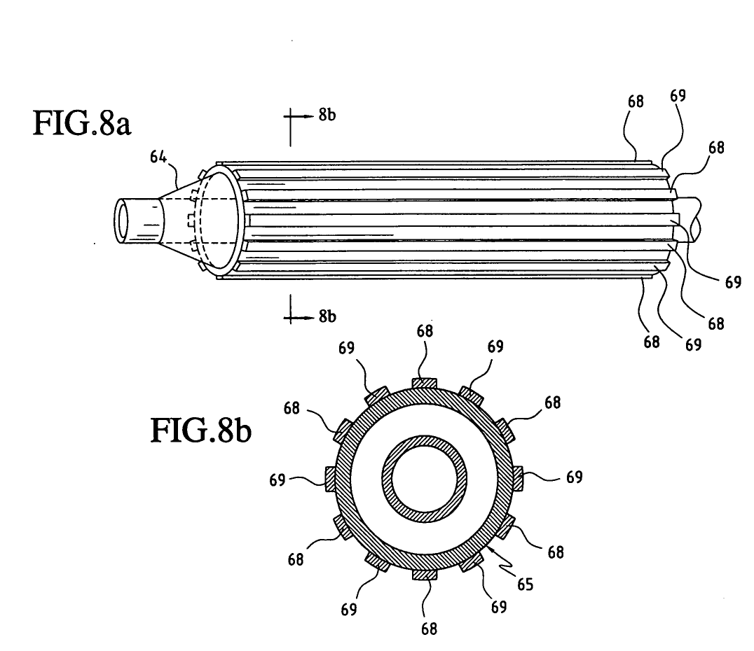

Referring now to FIGS. 7-8, another embodiment of the warming assembly is illustrated, designated generally as 60. In this embodiment the portion inserted through the patient's urethra is an RF heated warming catheter subassembly, designated generally as 62. The RF heated warming catheter subassembly 62 includes an RF heated tube assembly 64. An RF generating assembly is disposed within the electrically heated tube assembly. As can be seen in FIG. 8a, in the embodiment illustrated, the RF generating assembly includes alternating RF strips 68 and ground strips 69. This spacing of the strips provides a desired heating effect. The RF strips 68 are connected to an electrical generator that originates alternating current delivered at high frequency via the spaced strips 68 in the RF heated tube assembly. Ions in the tissue follow the alternating current delivered by the RF heated tube assembly. The ionic agitation causes frictional heating resulting in a warming. Other types of RF electrical generating systems are known in the medical device industry and are manufactured by various companies such as, for example, Valleylab, Inc., a division of Tyco Healthcare Group LP, Boulder, Colo. Valleylab is the assignee of U.S. Pat. No. 5,772,659, entitled “Electrosurgical Generator Power Control Circuit and Method”; and, U.S. Pat. No. 6,033,399, entitled “Electrosurgical Generator With Adaptive Power Control.” These units are generally used to cut and coagulate tissue of a patient. However, using lower power levels, the units may be used to warm tissue.

As in the previous embodiments, the RF heated warming catheter subassembly 62 preferably includes an elongated opening 66 to provide, for example, access for an endoscope and/or drainage for bladder fluid.

Referring again to FIG. 1, in an alternative method, a suprapubic suction tube 70 may be inserted into the bladder 16 of the patient 12. The suction tube 70 is operated to expel bladder fluid from the bladder 16 during the delivering of heat exchange fluid through the warming assembly 20.

Thus, while the preferred embodiments of the devices and methods have been described in reference to the environment in which they were developed, they are merely illustrative of the principles of the invention. Other embodiments and configurations may be devised without departing from the spirit of the invention and the scope of the appended claims.

1. A method for warming the urethra of a patient during ablative surgery, comprising the steps of:
a) inserting at least one ablative surgical device into a prostate region of the patient;
b) inserting a Joule-Thomson warming assembly through the patient's urethra and at least to the bladder neck; and,
c) operating said Joule-Thomson warming assembly to warm an outer surface thereof during operation of said at least one ablative surgical device;
wherein said urethra is warmed by said outer surface of said Joule-Thomson warming assembly to preserve living tissue thereof.

2. The method of claim 1, wherein said step of Inserting at least one ablative surgical device into a prostate region of the patient, comprises inserting at least one cryosurgical probe.

3. The method of claim 1, wherein said step of inserting a Joule-Thomson warming assembly comprises inserting a Joule-Thomson warming subassembly, comprising;
a) a tube assembly having a closed distal end portion, said tube assembly having said outer surface thereon; and,
b) a finned tube coiled heat exchanger disposed within said tube assembly, said heat exchanger having a Joule-Thomson nozzle on a distal end thereof and a high pressure gas inlet at a proximal end thereof, said finned tube coiled heat exchanger having a plurality of windings with interstitial gaps between the windings to provide an outlet path for hot gas expelled from said Joule-Thomson nozzle, wherein during operation said windings provide heat transfer from said outlet path to inlet gases for enhanced efficiency, the outer surface of said tube assembly being heated to provide warming of the urethra.

4. The method of claim 1, wherein said step of inserting a Joule-Thomson warming assembly comprises inserting a Joule-Thomson warming subassembly, comprising:
a) a tube assembly having a closed distal end portion, said tube assembly having said outer surface thereon, said tube assembly further including an elongated opening along a central axis of said tube assembly; and,
b) a finned tube coiled heat exchanger disposed within said tube assembly, said heat exchanger having a Joule-Thomson nozzle on a distal end thereof and a high pressure gas inlet at a proximal end thereof, said finned tube coiled heat exchanger having a plurality of windings with interstitial gaps between the windings to provide an outlet path for hot gas expelled from said Joule-Thomson nozzle, wherein during operation said windings providing heat transfer from said outlet path to inlet gases for enhanced efficiency, the outer surface of said tube assembly being heated to provide warming of the urethra, and wherein said central opening is in fluid isolation from both said gases flowing in said finned tube coiled heat exchanger and said outlet gases.

5. The method of claim 1, wherein said step of inserting a Joule-Thomson warming assembly comprises inserting a Joule-Thomson warming subassembly, comprising:
a) a tube assembly having a closed distal end portion, said tube assembly having said outer surface thereon, said tube assembly further including an elongated opening along a central axis of said tube assembly; and,
b) a finned tube coiled heat exchanger disposed within said tube assembly, said heat exchanger having a Joule-Thomson nozzle on a distal end thereof and a high pressure gas inlet at a proximal end thereof, said finned tube coiled heat exchanger having a plurality of windings with interstitial gaps between the windings to provide an outlet path for hot gas expelled from said Joule-Thomson nozzle, wherein during operation said windings providing heat transfer from said outlet path to inlet gases for enhanced efficiency, the outer surface of said tube assembly being heated to provide warming of the urethra, and wherein said central opening is in fluid isolation from both said gases flowing in said finned tube coiled heat exchanger and said outlet gases, said central opening providing access for an endoscope.

6. The method of claim 1, wherein said step of inserting a Joule-Thomson warming assembly comprises inserting a Joule-Thomson warming subassembly, comprising:
c) a tube assembly having a closed distal end portion, said tube assembly having said outer surface thereon, said tube assembly further including an elongated opening along a central axis of said tube assembly; and,
d) a finned tube coiled heat exchanger disposed within said tube assembly, said heat exchanger having a Joule-Thomson nozzle on a distal end thereof and a high pressure gas inlet at a proximal end thereof, said finned tube coiled heat exchanger having a plurality of windings with interstitial gaps between the windings to provide an outlet path for hot gas expelled from said Joule-Thomson nozzle, wherein during operation said windings providing heat transfer from said outlet path to inlet gases for enhanced efficiency, the outer surface of said tube assembly being heated to provide warming of the urethra, and wherein said central opening is in fluid isolation from both said gases flowing in said finned tube coiled heat exchanger and said outlet gases, said central opening providing access for a drainage tube.

7. The method of claim 1, wherein said step of operating said Joule-Thomson warming assembly comprises utilizing helium.

8. The method of claim 1, wherein said step of inserting said Joule-Thomson warming assembly comprises inserting a Joule-Thomson warming assembly, comprising:
a) an inlet subassembly for receiving an inlet flow of warming fluid;
b) a Joule-Thomson warming subassembly connected to said inlet subassembly for receiving said warming fluid from said inlet subassembly and providing warming of said urethra during operation;
c) a connector element for connecting said inlet subassembly to said Joule-Thomson warming subassembly; and,
d) an outlet subassembly for receiving warming fluid from said Joule-Thomson warming subassembly and providing an outlet flow thereof.

9. A method for warming the urethra of a patient during ablative surgery, comprising the steps of:
a) inserting at least one ablative surgical device into a prostate region of the patient:
b) inserting an electrically generated warming assembly through the patient's urethra and at least to the bladder neck; and,
c) operating said electrically generated warming assembly to warm an outer surface thereof during operation of said at least one ablative surgical device; wherein said urethra is warmed by said outer surface of said electrically generated warming assembly to preserve living tissue thereof.

10. The method of claim 9, wherein said step of inserting at least one ablative surgical device into a prostate region of the patient, comprises inserting at least one cryosurgical probe.

11. The method of claim 9, wherein said electrically generated warming assembly comprises an electrically generated tube assembly including an elongated opening along a central axis thereof for providing fluid drainage or access for an endoscope.

12. The method of claim 9, wherein said step of inserting an electrically generated warming assembly, comprises inserting an electrical coil heated warming catheter subassembly, comprising:
a) an electrical coil heated tube assembly having a closed distal end portion, said tube assembly having said outer surface thereon; and,
b) an electrical coil assembly disposed within said electrically heated tube assembly.

13. The method of claim 12, wherein said electrical coil heated tube assembly includes an elongated opening along a central axis thereof for providing fluid drainage or access for an endoscope.

14. The method of claim 9, wherein said step of inserting an electrically generated warming assembly, comprises inserting a microwave heated warming catheter subassembly, comprising,
a) a microwave heated tube assembly having a closed distal end portion, said tube assembly having said outer surface thereon; and,
b) a microwave generating assembly disposed within said microwave heated tube assembly.

15. The method of claim 14, wherein said microwave heated tube assembly includes an elongated opening along a central axis thereof for providing fluid drainage or access for an endoscope.

16. The method of claim 9, wherein said step of inserting an electrically generated warming assembly, comprises inserting an RF heated warming catheter subassembly, comprising:
a) an RF heated tube assembly having a closed distal end portion, said tube assembly having said outer surface thereon; and,
b) an RF generating assembly operatively associated with said RF heated tube assembly.

17. The method of claim 16, wherein said RF heated tube assembly includes an elongated opening along a central axis thereof for providing fluid drainage or access for an endoscope.

18. A warming assembly for warming the urethra of a patient during ablative surgery, comprising:
a Joule-Thomson warming assembly, comprising a Joule-Thomson warming subassembly, comprising:
a) a tube assembly having a closed distal end portion, said tube assembly having an outer surface thereon; and
b) a finned tube coiled heat exchanger disposed within said tube assembly, said heat exchanger having a Joule-Thomson nozzle on a distal end thereof and a high pressure gas inlet at a proximal end thereof, said finned tube coiled heat exchanger having a plurality of windings with interstitial gaps between the windings to provide an outlet path for hot gas expelled from said Joule-Thomson nozzle, wherein during operation said windings provide heat transfer from said outlet path to inlet gases for enhanced efficiency, the outer surface of said tube assembly being heated to provide warming of said urethra.

19. A warming assembly for warming the urethra of a patient during ablative surgery, comprising;
a Joule-Thomson warming assembly, comprising a Joule-Thomson warming subassembly, comprising:
a) a tube assembly having a closed distal end portion, said tube assembly having an outer surface thereon, said tube assembly further including an elongated opening along a central axis of said tube assembly; and
b) a finned tube coiled heat exchanger disposed within said tube assembly, said heat exchanger having a Joule-Thomson nozzle on a distal end thereof and a high pressure gas inlet at a proximal end thereof, said finned tube coiled heat exchanger having a plurality of windings with interstitial gaps between the windings to provide an outlet path for hot gas expelled from said Joule-Thomson nozzle, wherein during operation said windings providing heat transfer from said outlet path to inlet gases for enhanced efficiency, the outer surface of said tube assembly being heated to provide warming of said urethra, and wherein said central opening is in fluid isolation from both said gases flowing in said finned tube coiled heat exchanger and said outlet gases.

20. The warming assembly of claim 19, wherein said central opening provides access for an endoscope.

21. The warming assembly of claim 19, wherein said central opening provides access for a drainage tube.

22. A warming assembly for warming the urethra of a patient during ablative surgery, comprising:
a Joule-Thomson warming assembly, comprising a Joule-Thomson warming subassembly, comprising:
a) an Inlet subassembly for receiving an inlet flow of warming fluid;
b) a Joule-Thomson warming subassembly connected to said inlet subassembly for receiving said warming fluid from said inlet subassembly and providing warming of said urethra during operation;
c) a connector element for connecting said inlet subassembly to said Joule-Thomson warming subassembly; and,
d) an outlet subassembly for receiving warming fluid from said Joule-Thomson warming subassembly.

23. A warming assembly for warming the urethra of a patient during ablative surgery, comprising:
an electrically generated warming assembly, comprising an electrical coil heated warming catheter subassembly, comprising:
a) an electrical coil heated tube assembly having a closed distal end portion, said tube assembly having said outer surface thereon; and,
b) an electrical coil assembly disposed within said electrically heated tube assembly.

24. The warming assembly of claim 23, wherein said electrical coil heated tube assembly includes an elongated opening along a central axis thereof for providing fluid drainage or access for an endoscope.

25. A warming assembly for warming the urethra of a patient during ablative surgery, comprising:
an electrically generated warming assembly, comprising an microwave heated warming catheter subassembly, comprising:
a) a microwave heated tube assembly having a dosed distal end portion, said tube assembly having said outer surface thereon; and,
b) a microwave generating assembly disposed within said microwave heated tube assembly.

26. The method of claim 25, wherein said microwave heated tube assembly includes an elongated opening along a central axis thereof for providing fluid drainage or access for an endoscope.

27. A warming assembly for warming the urethra of a patient during ablative surgery, comprising:
an electrically generated warming assembly, comprising an RF heated warming catheter subassembly, comprising:
a) a microwave heated tube assembly having a closed distal end portion, said tube assembly having said outer surface thereon; and,
b) a microwave generating assembly disposed within said microwave heated tube assembly.

28. The warming assembly of claim 27, wherein said microwave heated tube assembly includes an elongated opening along a central axis thereof for providing fluid drainage or access for an endoscope.
